(19) United States
(12) Patent Application Publication
(10) Pub. No.: US 2003/0145349 A1
(43) Pub. Date: Jul. 31, 2003

(54) Plant transcription coactivators with histone acetyl transferase activity

(76) Inventors: Joan Tellefsen Odell, Unionville, PA (US); Zhan-Bin Liu, West Chester, PA (US); Hajime Sakai, Newark, DE (US); Rebecca E. Cahoon, Webster Groves, MO (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/354,804

(22) Filed: Jan. 30, 2003

Related U.S. Application Data
(63) Continuation of application No. 09/424,977, filed on Dec. 2, 1999.
Application No. 09/424,977 is a 371 of international application No. PCT/US98/12071, filed on Jun. 11, 1998.
(60) Provisional application No. 60/049,408, filed on Jun. 12, 1997.

(51) Int. Cl. A01H 1/00; C12N 15/82; C07H 21/04; C12N 5/04

(52) U.S. Cl. 800/278; 435/468; 435/320.1; 536/23.2; 435/419

(57) ABSTRACT

This invention relates to isolated nucleic acid fragments encoding all or a substantial portion of a maize, rice, or wheat histone acetyltransferase protein. The invention also relates to the construction of chimeric genes encoding all or a portion of a maize, rice, or wheat histone acetyltransferase protein, in sense or antisense orientation, wherein expression of the chimeric gene results in production of altered levels of a maize, rice, or wheat histone acetyltransferase protein in a transformed host cell. The invention also relates to targeting of the maize, rice, or wheat histone acetyltransferase protein to a novel promoter region by the addition of either a DNA-binding domain or a protein-protein interaction domain, to acetylate the core histone, weaken the interaction between core histones and DNA, open up chromatin structure, increase the efficiency of transcriptional initiation, thus leading to a higher level of gene expression.

This application is a continuation of U.S. patent application Ser. No. 09/424,977, filed Dec. 2, 1999, which is a 35 U.S.C. 371 filing of PCT/US98/12071, filed Jun. 11, 1998, which claims the benefit of U.S. Provisional Application No. 60/049,408, filed Jun. 12, 1997.

### FIELD OF THE INVENTION

This invention is in the field of plant molecular biology. More specifically, this invention pertains to nucleic acid fragments encoding coactivator proteins involved in regulation of gene expression in plants and seeds.

### BACKGROUND OF THE INVENTION

High level of gene expression requires specific interaction between transcription factors and their target sites. The efficiency of gene activation depends on the accessibility of these target sites to gene-specific transcription factors. In the case of genes located on nuclear chromosomes, the DNA containing the target sites is associated with histone proteins forming nucleosome arrays. There is a general correlation between the level of histone acetylation and the transcriptional activity of a chromosomal domain (Hebbes et al. (1994) EMBO J. 7: 1395-1402). It is believed that histone hyperacetylation directs an allosteric change in nucleosome conformation, destabilizes higher-order structure and renders nucleosomal DNA more accessible to transcription factors (Lee et al. (1993) Cell 72: 73-84; Garcia-Ramirez et al. (1995) J. Biol. Chem. 270: 17923-17928).

In yeast, GCN5 (yGCN5) is a transcriptional coactivator that enhances the activation of transcription by acidic activators such as GCN4, Gal4-VP16, and the HAP2-HAP3-HAP4 complex (Georgakopoulos, T. and Thireos, G. (1992) EMBO J. 11: 4145-4152). yGCN5 itself is also a histone acetyltransferase (Brownell et al. (1996) Cell 84: 843-851). It is proposed that yGCN5 can be recruited to a specific gene through selective interaction with a subset of transcription factors (Wolffe and Pruss, (1996) Cell 84: 817-819). It has also been shown that targeting yGCN5 to a promoter by fusing it to a heterologous DNA-binding domain leads to transcriptional activation in yeast, most probably due to the histone acetyltransferase activity of yGCN5 (Candau et al. (1997) EMBO J. 16:555-565). Two human yGCN5 homologs (P/CAF and hGCN5) have been isolated. Both have histone acetyltransferase activities (Yang et al. (1996) Nature 382:319-324). Lastly, a DNA sequence encoding an Arabidopsis thaliana histone acetyltransferase protein has recently been identified and isolated (GenBank Accession No. AF031958).

### SUMMARY OF THE INVENTION

The instant invention relates to isolated nucleic acid fragments encoding plant histone acetyltransferase proteins that may be involved in regulation of gene expression. More particularly, this invention concerns isolated nucleic acid fragments encoding maize, rice, and wheat histone acetyltransferase proteins. In addition, this invention relates to nucleic acid fragments that are complementary to nucleic acid fragments encoding the maize, rice and wheat histone acetyltransferase proteins.

In another embodiment, the instant invention relates to a chimeric gene that comprises a nucleic acid fragment encoding a maize, rice, or wheat histone acetyltransferase protein, or to a chimeric gene that comprises a nucleic acid fragment that is complementary to a nucleic acid fragment encoding a maize, rice, or wheat histone acetyltransferase protein, the nucleic acid fragment being operably linked to suitable regulatory sequences, wherein expression of the chimeric gene results in production of levels of the encoded protein in transformed host cells that are altered (i.e., increased or decreased) relative to the levels produced in untransformed host cells.

In a further embodiment, the instant invention concerns a transformed host cell comprising in its genome a chimeric gene comprising a nucleic acid fragment encoding a maize, rice, or wheat histone acetyltransferase protein or a chimeric gene comprising a nucleic acid fragment that is complementary to the nucleic acid fragment encoding a maize, rice, or wheat histone acetyltransferase protein, the chimeric gene being operably linked to suitable regulatory sequences. Expression of the chimeric gene results in production of altered levels of protein encoded by the operably linked nucleic acid fragment in the transformed host cell. The transformed host cell can be of eukaryotic or prokaryotic origin, and include cells derived from higher plants and microorganisms. The invention also includes transformed plants that arise from transformed host cells of higher plants, and seeds derived from such transformed plants.

An additional embodiment of the instant invention concerns a method of altering the level of expression of a maize, rice, or wheat histone acetyltransferase protein in a transformed host cell comprising: a) transforming a host cell with a chimeric gene comprising a nucleic acid fragment encoding a maize, rice, or wheat histone acetyltransferase protein or a chimeric gene that comprises a nucleic acid fragment that is complementary to the nucleic acid fragment encoding a maize, rice, or wheat histone acetyltransferase protein; and b) growing the transformed host cell under conditions that are suitable for expression of the chimeric gene wherein expression of the chimeric gene results in production of altered levels of protein encoded by the operably linked nucleic acid fragment in the transformed host cell.

An addition embodiment of the instant invention concerns a method for obtaining a nucleic acid fragment encoding all or substantially all of an amino acid sequence encoding a maize, rice, or wheat histone acetyltransferase protein.

A further embodiment of the instant invention is a method for evaluating at least one compound for its ability to inhibit the activity of a maize, rice, or wheat histone acetyltransferase protein protein, the method comprising the steps of: (a) transforming a host cell with a chimeric gene comprising a nucleic acid fragment encoding a maize, rice, or wheat histone acetyltransferase protein, operably linked to suitable regulatory sequences; (b) growing the transformed host cell under conditions that are suitable for expression of the chimeric gene wherein expression of the chimeric gene results in production of the protein encoded by the operably linked nucleic acid fragment in the transformed host cell; (c) optionally purifying the protein expressed by the transformed host cell; (d) treating the protein with a compound to be tested; and (e) comparing the activity of the protein that has been treated with a test compound to the activity of an untreated protein, thereby selecting compounds with potential for inhibitory activity.

### BRIEF DESCRIPTION OF THE DRAWINGS AND SEQUENCE DESCRIPTIONS

The invention can be more fully understood from the following detailed description and the accompanying drawing and sequence descriptions which form a part of this application.

FIGS. 1A and 1B show a comparison of the amino acid sequences of the Saccharomyces cerevisiae GCN5 transcriptional coactivator (X68628), a human histone acetyltransferase (U57316) and the instant maize and wheat GCN5 protein homologs (maize GCN5 and w11n.pk0003.c2, respectively).

FIGS. 2A and 2B show a comparison of the amino acid sequence of the Arabidopsis thaliana histone acetyltransferase protein and the instant histone acetyltransferase homologs encoded by clones cep7.pk0001.a11, w11n.pk0003.c2, wr1.pk0045.f4 and r1r6.pk0084.c5.

The following sequence descriptions and sequence listings attached hereto comply with the rules governing nucleotide and/or amino acid sequence disclosures in patent applications as set forth in 37 C.F.R. §1.821-1.825.

SEQ ID NO:1 is the nucleotide sequence comprising the entire cDNA insert in clone cep7.pk0001.a11 encoding cGCN5, a maize homolog of the yeast GCN5 protein.

SEQ ID NO:2 is the deduced amino acid sequence of a portion of a of maize homolog of the yeast GCN5 derived from the nucleotide sequence of SEQ ID NO:1.

SEQ ID NO:3 is the nucleotide sequence comprising a portion of the cDNA insert in clone w11n.pk0003.c2 encoding wGCN5, a wheat homolog of the yeast GCN5 protein.

SEQ ID NO:4 is the deduced amino acid sequence of a portion of a wheat homolog of the yeast GCN5 derived from the nucleotide sequence of SEQ ID NO:3.

SEQ ID NO:5 is the amino acid sequence encoding the Saccharomyces cerevisiae GCN5 transcriptional coactivator having EMBL accession No. X68628.

SEQ ID NO:6 is the amino acid sequence encoding a human histone acetyltransferase having GenBank accession No. U57316.

SEQ ID NOs:7, 8 and 9 are the nucleotide sequences of the 5′ RACE primers GSP1, GSP2 and GSP3, respectively, used in the 5′ RACE protocol in order to isolate a nucleic acid fragment encoding the 5′ nucleotide sequence of the maize homolog of the yeast GCN5.

SEQ ID NOs:10 and 11 are the nucleotide sequences of the 5′ RACE Abridged Anchor primer and the Abridged Universal Amplification primer, respectively, used in the 5′ RACE protocol.

SEQ ID NO:12 is the nucleotide sequence of the contig assembled from SEQ ID NO:1 and the nucleotide sequence information obtained from the 5′ RACE protocol encoding a portion of a maize homolog of the yeast GCN5 protein.

SEQ ID NO:13 is the deduced amino acid sequence derived from the nucleotide sequence set forth in SEQ ID NO:13.

SEQ ID NO:14 is the nucleotide sequence comprising a portion of the cDNA insert in clone r1r6.pk0084.c5 encoding a rice histone acetyltransferase.

SEQ ID NO:15 is the deduced amino acid sequence of a histone acetyltransferase derived from the nucleotide sequence of SEQ ID NO:14.

SEQ ID NO:16 is the nucleotide sequence comprising a portion of the cDNA insert in clone wr1.pk0045.f4 encoding a wheat histone acetyltransferase.

SEQ ID NO:17 is the deduced amino acid sequence of a histone acetyltransferase derived from the nucleotide sequence of SEQ ID NO:16.

SEQ ID NO:18 is the amino acid sequence encoding an Arabidopsis thaliana histone acetyltransferase set forth in GenBank Accession No. AFO31958.

The Sequence Descriptions contain the one letter code for nucleotide sequence characters and the three letter codes for amino acids as defined in conformity with the IUPAC-IYUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

### DETAILED DESCRIPTION OF THE INVENTION

The amino acid sequence similarity between the instant maize, rice, and wheat histone acetyltransferase proteins and the Arabidopsis thaliana histone acetyltransferase and yGCN5 proteins indicates that the maize, rice, or wheat histone acetyltransferase proteins may function as transcriptional coactivators. The maize, rice or wheat huistone acetyltransferese proteins may be used to reduce expression of specific genes whose promoters are normally regulated by a histone acetyltransferase activity, using antisense or co-suppression technology. The plant proteins may also be used to enhance gene expression of those genes whose promoters are normally targeted by the transcription factors that histone acetyltransferase proteins normally interact with.

Alternatively, the maize, rice, or wheat histone acetyltransferase protein coactivation function can be targeted to a novel promoter region by the addition of either a DNA binding domain or a protein-protein interaction domain. The instant maize, rice, or wheat histone acetyltransferase proteins can be fused to a very defined DNA-binding domain, such as, but not limited to, a bacterial lexA DNA binding domain, a yeast Gal4 DNA-binding domain or a DNA binding domain from a plant transcription factor. For example, it has also been shown that targeting yGCN5 to a promoter by fusing it to a heterologous DNA-binding domain leads to transcriptional activation in yeast, most probably due to the histone acetyltransferase activity of yGCN5 (Candau et al. (1997) EMBO J. 16:555-565). Alternatively, a synthetic promoter can be designed to contain multiple copies of a target site which is necessary for the specific binding by either the lexA, Gal4 or plant DNA binding domain. By using this approach, the maize, rice, or wheat histone acetyltransferase protein can be specifically targeted to the engineered synthetic promoter to acetylate the core histones, weaken the interaction between core histones and DNA, open up chromatin structure, and increase the efficiency of transcriptional initiation, thus leading to a higher level of gene expression. Additionally, maize, rice, or wheat histone acetyltransferase protein can be fused to a transcription factor that already includes its own DNA binding domain in order to target the coactivator. Besides DNA-binding domains, the maize, rice, or wheat histone acetyltransferase protein can also be fused to other transcription regulatory proteins, such as mediators. Normally, these mediators do not bind to DNA directly and are recruited to their target sites by interaction with other DNA-binding proteins. By fusing the maize, rice, or wheat histone acetyltransferase protein to these mediators, the histone acetyltransferase activity can be targeted to specific regulatory elements through the interaction between the mediators and other DNA-binding proteins. Considering the histone acetyltransferase activity of yGCN5 and human GCN5 homologs, the maize, rice, or wheat histone acetyltransferase proteins can provide a tool to enhance trait gene expression by targeted histone acetylation.

In the context of this disclosure, a number of terms shall be utilized. As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA. As used herein, “contig” refers to an assemblage of overlapping nucleic acid sequences to form one contiguous nucleotide sequence. For example, several DNA sequences can be compared and aligned to identify common or overlapping regions. The individual sequences can then be assembled into a single contiguous nucleotide sequence.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotides that do not substantially affect the functional properties of the resulting transcript vis-a-vis the ability to mediate alteration of gene expression by antisense or co-suppression technology or alteration of the functional properties of the resulting protein molecule. It is therefore understood that the invention encompasses more than the specific exemplary sequences.

For example, it is well known in the art that antisense suppression and co-suppression of gene expression may be accomplished using nucleic acid fragments representing less than the entire coding region of a gene, and by nucleic acid fragments that do not share 100% sequence identity with the gene to be suppressed. Moreover, alterations in a gene which result in the production of a chemically equivalent amino acid at a given site, but do not effect the functional properties of the encoded protein, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the protein molecule would also not be expected to alter the activity of the protein. Each of the proposed modifications is well within the routine skill in the art, as is determination of retention of biological activity of the encoded products. Moreover, the skilled artisan recognizes that substantially similar nucleic acid sequences encompassed by this invention are also defined by their ability to hybridize, under stringent conditions (0.1×SSC, 0.1% SDS, 65° C.), with the sequences exemplified herein. Preferred substantially similar nucleic acid fragments of the instant invention are those nucleic acid fragments whose DNA sequences are 80% identical to the coding sequence of the nucleic acid fragments reported herein. More preferred nucleic acid fragments are 90% identical to the coding sequence of the nucleic acid fragments reported herein. Most preferred are nucleic acid fragments that are 95% identical to the coding sequence of the nucleic acid fragments reported herein.

A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www.ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to afford specific identification and/or isolation of a nucleic acid fragment comprising the sequence. The instant specification teaches partial or complete amino acid and nucleotide sequences encoding one or more particular plant proteins. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing, as well as substantial portions of those sequences as defined above.

“Codon degeneracy” refers to divergence in the genetic code permitting variation of the nucleotide sequence without effecting the amino acid sequence of an encoded polypeptide. Accordingly, the instant invention relates to any nucleic acid fragment that encodes all or a substantial portion of the amino acid sequences set forth in SEQ ID NOs:2, 4, 13, 15 and 17. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that encodes a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a DNA sequence capable of controlling the expression of a coding sequence or functional RNA. In general, a coding sequence is located 3′ to a promoter sequence. The promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. New promoters of various types useful in plant cells are constantly being discovered; numerous examples may be found in the compilation by Okamuro and Goldberg, (1989) Biochemistry of Plants 15:1-82. It is further recognized that since in most cases the exact boundaries of regulatory sequences have not been completely defined, DNA fragments of different lengths may have identical promoter activity.

The “translation leader sequence” refers to a DNA sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Molecular Biotechnology 3:225).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript. Alternatively, the RNA transcript may be an RNA sequence derived from posttranscriptional processing of the primary transcript; this is referred to as the mature RNA. “Messenger RNA” (mRNA) refers to the RNA that is without introns and that can be translated into protein by the cell. “cDNA” refers to a double-stranded DNA that is complementary to and derived from mRNA. “Sense” RNA refers to an RNA transcript that includes the mRNA and so can be translated into protein by the cell. “Antisense RNA” refers to a RNA transcript that is complementary to all or part of a target primary transcript or mRNA and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that is not translated yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020).

“Altered levels” refers to the production of gene product(s) in transgenic organisms in amounts or proportions that differ from that of normal or non-transformed organisms.

“Transformation” refers to the transfer of a nucleic acid fragment into the genome of a host organism, resulting in genetically stable inheritance. Host organisms containing the transformed nucleic acid fragments are referred to as “transgenic” organisms. Examples of methods of plant transformation include Agrobacterium-mediated transformation (De Blaere et al. (1987) Meth. Enzymol. 143:277) and particle-accelerated or “gene gun” transformation technology (Klein et al. (1987) Nature (London) 327:70-73; U.S. Pat. No. 4,945,050).

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Maniatis”).

This invention relates to maize cDNAs with homology to the yeast GCN5 transcritional coactivator, human histone acetyltransfease and Arabidopsis thaliana histone acetyltransferase protein. The invention also relates to rice and wheat cDNAs with homology to Arabidopsis thaliana histone acetyltransferase protein. The instant maize, rice and wheat cDNAs have been isolated and identified by comparison of random plant cDNA sequences to the GenBank database using the BLAST algorithms well known to those skilled in the art. The nucleotide sequence of a maize histone acetyltransferase protein is provided in SEQ ID NO:1, and the deduced amino acid sequence is provided in SEQ ID NO:2. The nucleotide sequence of a wheat histone acetyltransferase protein is provided in SEQ ID NO:3, and the deduced amino acid sequence is provided in SEQ ID NO:4. The nucleotide sequence of a wheat histone acetyltransferse protein is provided in SEQ ID NO:16, and the deduced amino acid sequence is provided in SEQ ID NO:17. This sequence appears to be more 5′ than SEQ ID NO:3 but overlapping regions of homology were not long enough to form a contig. Lastly, the nucleotide sequence of a rice histone acetyltransferse protein is provided in SEQ ID NO:14, and the deduced amino acid sequence is provided in SEQ ID NO:15. Homologs of histone acetyltransferase proteins from other plants can now be identified by comparison of random cDNA sequences to the maize, rice and wheat histone acetlytransferase sequences provided herein.

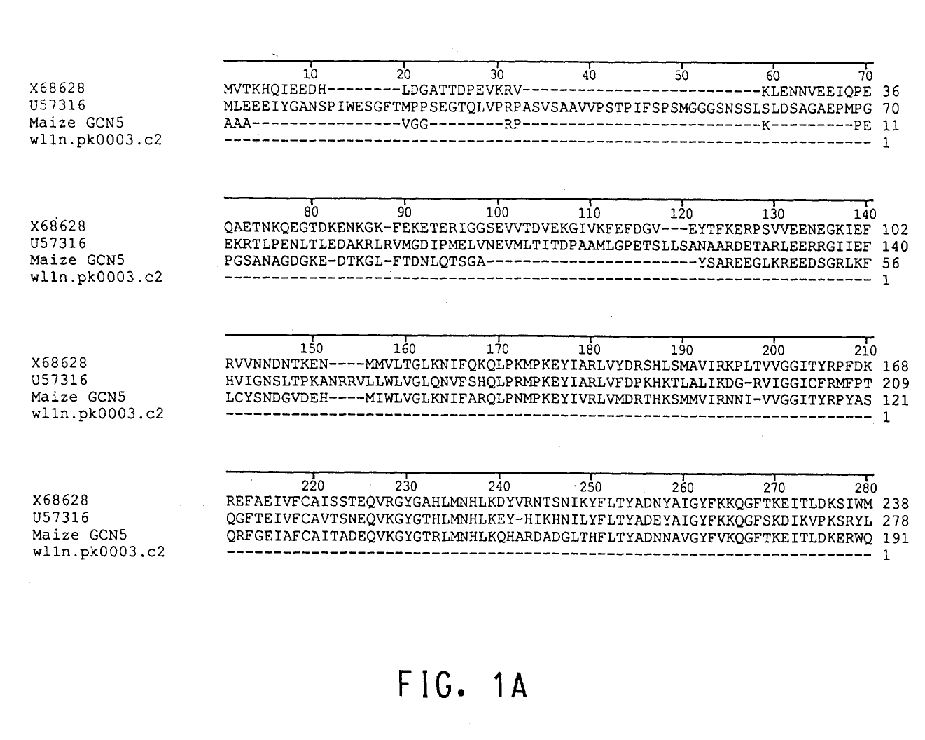

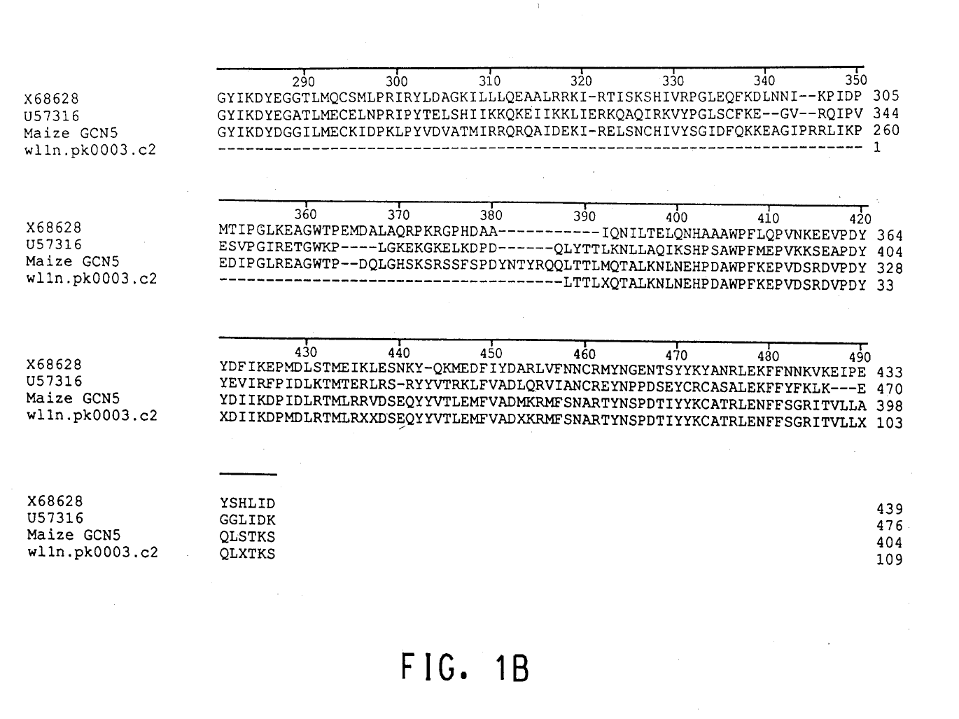

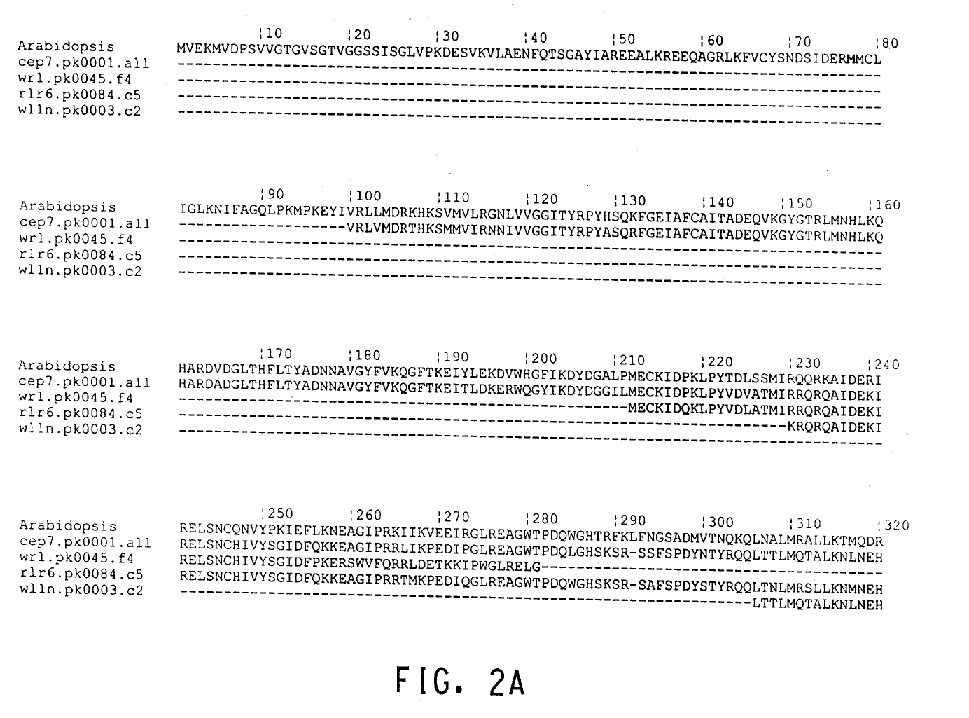

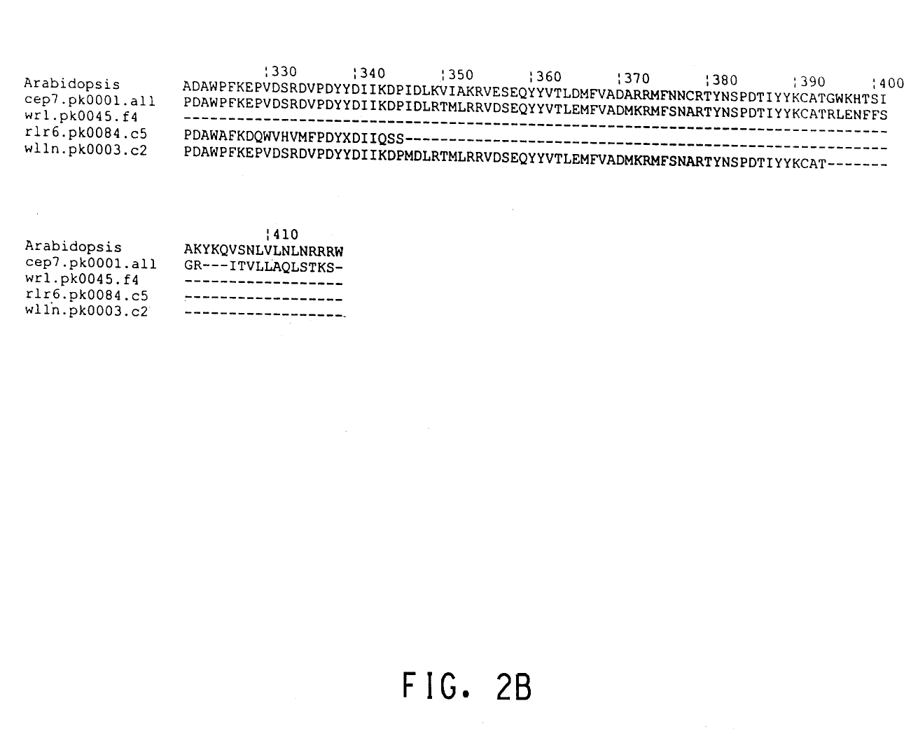

The full insert of cDNA clone cep7.pk0001.a11 encoding the maize homolog of yGCN5 has been completely sequenced. Amino acid sequence comparison indicates that there is 45.9% sequence identity between this maize homolog and yGCN5 (FIG. 1). It is even more striking that all the potential important domains for acetyltransferase activity (domain 1, 2, 3, 4) and protein interaction (domain 5: bromodomain) are extremely conserved. Based on the high sequence homology, it is believed that this maize clone encodes a plant homolog of yGCN5. This is the first indication that there is a similar GCN5-mediated gene activation system in plants.

Likewise, the insert in EST clone w11n.pk0003.c2 appears to encode the 3′coding region of a wheat GCN5 homolog. At the amino acid level, this wheat peptide is approximately 80% identical to the maize GCN5 encoded by cDNA clone cep7.pk0001.a11. Nucleotide identity between the wheat and maize cDNAs is approximately 62%. Sequence alignments and percent identity calculations were performed by the Jotun Hein method using the Megalign program of DNAStar™ sequence analysis software (DNASTAR Inc. 1228 South Park Street, Madison Wis., 53715).

Lastly, the inserts in EST clones r1r6.pk0084.c5 and wr1.pk0045.f4 appear to encode the 3′ coding regions of a rice and wheat histone acetyltransferase homolog. At the amino acid level, the histone acetyltransferases encoded by the cDNAs of r1r6.pk0084.c5 wr1.pk0045.f4 are 49% and 52% (respectively) identical to the Arabidopsis thaliana histone acetyltransferase peptide set forth in GenBank Accession No. AF031958.

The nucleic acid fragments of the instant invention may be used to isolate cDNAs and genes encoding other gene homologs of histone acetyltransferase proteins from the same or other plant species. Isolation of homologous genes using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other plant homologs of histone acetyltransferase protein, either as cDNAs or genomic DNAs, could be isolated directly by using all or a portion of the instant nucleic acid fragments as DNA hybridization probes to screen libraries from any desired plant employing methodology well known to those skilled in the art. Specific oligonucleotide probes based upon the instant nucleic acid sequences can be designed and synthesized by methods known in the art (Maniatis). Moreover, the entire sequences can be used directly to synthesize DNA probes by methods known to the skilled artisan such as random primers DNA labeling, nick translation, or end-labeling techniques, or RNA probes using available in vitro transcription systems. In addition, specific primers can be designed and used to amplify a part of or full-length of the instant sequences. The resulting amplification products can be labeled directly during amplification reactions or labeled after amplification reactions, and used as probes to isolate full length cDNA or genomic fragments under conditions of appropriate stringency. Genomic fragments can be isolated that include the promoter region that directs expression of the maize, rice, or wheat histone acetyltransferase protein protein. This promoter may be prepared as a DNA fragment including regulatory elements with or without the untranslated leader and used in expression of other coding regions or for co-suppression.

In addition, two short segments of the instant nucleic acid fragments may be used in polymerase chain reaction protocols to amplify longer nucleic acid fragments encoding homologous genes from DNA or RNA. The polymerase chain reaction may also be performed on a library of cloned nucleic acid fragments wherein the sequence of one primer is derived from the instant nucleic acid fragments, and the sequence of the other primer takes advantage of the presence of the polyadenylic acid tracts to the 3′ end of the mRNA precursor encoding plant genes. Alternatively, the second primer sequence may be based upon sequences derived from the cloning vector. For example, the skilled artisan can follow the RACE protocol (Frohman et al., (1988) PNAS USA 85:8998) to generate cDNAs by using PCR to amplify copies of the region between a single point in the transcript and the 3′ or 5′ end. Primers oriented in the 3′ and 5′ directions can be designed from the instant sequences. Using commercially available 3′ RACE or 5′ RACE systems (BRL), specific 3′ or 5′ cDNA fragments can be isolated (Ohara et al., (1989) PNAS USA 86:5673; Loh et al., (1989) Science 243:217). Products generated by the 3′ and 5′ RACE procedures can be combined to generate full-length cDNAs (Frohman, M. A. and Martin, G. R., (1989) Techniques 1: 165). Further DNA sequence of the 5′ end of the maize homolog of the yeast GCN5 was obtained using the 5′ RACE protocol. The sequence of cDNA clone cep7.pk0001.a11 and the 5′ RACE sequence was used to assemble a contiguous DNA sequence for a portion of the maize homolog of the yeast GCN5, and is presented as SEQ ID NO:12.

Availability of the instant nucleotide and deduced amino acid sequences facilitates immunological screening of cDNA expression libraries. Synthetic peptides representing portions of the instant amino acid sequences may be synthesized. These peptides can be used to immunize animals to produce polyclonal or monoclonal antibodies with specificity for peptides or proteins comprising the amino acid sequences. These antibodies can be then be used to screen cDNA expression libraries to isolate full-length cDNA clones of interest (Lerner, R. A. (1984) Adv. Immunol. 36:1; Maniatis).

The nucleic acid fragments of the instant invention may be used to create transgenic plants in which the disclosed maize, rice and wheat histone acetyltransferase proteins are present at higher or lower levels than normal or in cell types or developmental stages in which it is not normally found. This would have the effect of altering the level of histone acetyltransferase activity in those cells.

Overexpression of maize, rice or wheat histone acetyltransferase proteins may be accomplished by first constructing a chimeric gene in which the coding region is operably linked to a promoter capable of directing expression of a gene in the desired tissues at the desired stage of development. For reasons of convenience, the chimeric gene may comprise a promoter sequence and translation leader sequence derived from the same gene. A 3′ non-coding sequence encoding a transcription termination signal may also be provided. The instant chimeric gene may also comprise one or more introns in order to facilitate gene expression.

Plasmid vectors comprising the instant chimeric gene can then constructed. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, Western analysis of protein expression, or phenotypic analysis.

For some applications, it may be desirable to reduce or eliminate expression of the genes encoding the instant maize, rice and wheat histone acetyltransferase proteins. In order to accomplish this, chimeric genes designed for co-suppression of the instant maize, rice and wheat histone acetyltransferase genes can be constructed by linking the genes or gene fragments encoding the maize, rice, or wheat histone acetyltransferase proteins to plant promoter sequences. Alternatively, chimeric genes designed to express antisense RNA for all or part of the instant nucleic acid fragments can be constructed by linking the genes or gene fragments in reverse orientation to plant promoter sequences. Either the co-suppression or antisense chimeric genes could be introduced into plants via transformation wherein expression of the corresponding endogenous genes are reduced or eliminated.

The instant plant histone acetyltransferase proteins (or portions thereof) may be produced in heterologous host cells, particularly in the cells of microbial hosts, and can be used to prepare antibodies to the histone acetyltransferase proteins by methods well known to those skilled in the art. The antibodies are useful for detecting maize, rice and wheat histone acetyltransferase proteins in situ in cells or in vitro in cell extracts. Preferred heterologous host cells for production of the instant histone acetyltransferase proteins are microbial hosts. Microbial expression systems and expression vectors containing regulatory sequences that direct high level expression of foreign proteins are well known to those skilled in the art. Any of these could be used to construct chimeric genes for production of the instant maize, rice and wheat histone acetyltransferase proteins. These chimeric genes could then be introduced into appropriate microorganisms via transformation to provide high level expression of maize, rice and wheat histone acetyltransferase proteins. An example of a vector for high level expression of the maize, rice and wheat acetyltransferase proteins in a bacterial host is provided (Example 7).

Additionally, the instant histone acetyltransferase proteins can be used as targets to facilitate design and/or identification of inhibitors of the protein that may be useful as herbicides. This is desirable because the protein described plays a key role in regulation of gene expression. Accordingly, inhibition of the activity of the proteins described herein could lead to inhibition of gene expression sufficient to inhibit plant growth. Thus, the instant maize, rice and wheat histone acetyltransferase proteins could be appropriate for new herbicide discovery and design.

All or a portion of the nucleic acid fragments of the instant invention may also be used as probes for genetically and physically mapping the genes that they are a part of, and as markers for traits linked to expression of the instant maize, rice and wheat histone acetyltransferase proteins. Such information may be useful in plant breeding in order to develop lines with desired phenotypes.

For example, the instant nucleic acid fragments may be used as restriction fragment length polymorphism (RFLP) markers. Southern blots (Maniatis) of restriction-digested plant genomic DNA may be probed with the nucleic acid fragments of the instant invention. The resulting banding patterns may then be subjected to genetic analyses using computer programs such as MapMaker (Lander et at., (1987) Genomics 1: 174-181) in order to construct a genetic map. In addition, the nucleic acid fragments of the instant invention may be used to probe Southern blots containing restriction endonuclease-treated genomic DNAs of a set of individuals representing parent and progeny of a defined genetic cross. Segregation of the DNA polymorphisms is noted and used to calculate the position of the instant nucleic acid sequence in the genetic map previously obtained using this population (Botstein, D. et al., (1980) Am. J. Hum. Genet. 32:314-331).

The production and use of plant gene-derived probes for use in genetic mapping is described in R. Bernatzky, R. and Tanksley, S. D. (1986) Plant Mol. Biol. Reporter 4(1):37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

Nucleic acid probes derived from the instant nucleic acid sequences may also be used for physical mapping (i.e., placement of sequences on physical maps; see Hoheisel, J. D., et al., In: Nonmammalian Genomic Analysis: A Practical Guide, Academic press 1996, pp. 319-346, and references cited therein).

In another embodiment, nucleic acid probes derived from the instant nucleic acid sequences may be used in direct fluorescence in situ hybridization (FISH) mapping. Although current methods of FISH mapping favor use of large clones (several to several hundred KB), improvements in sensitivity may allow performance of FISH mapping using shorter probes.

A variety of nucleic acid amplification-based methods of genetic and physical mapping may be carried out using the instant nucleic acid sequences. Examples include allele-specific amplification, polymorphism of PCR-amplified fragments (CAPS), allele-specific ligation, nucleotide extension reactions, Radiation Hybrid Mapping and Happy Mapping. For these methods, the sequence of a nucleic acid fragment is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods. Such information may be useful in plant breeding in order to develop lines with desired starch phenotypes.

Loss of function mutant phenotypes may be identified for the instant cDNA clones either by targeted gene disruption protocols or by identifying specific mutants for these genes contained in a maize population carrying mutations in all possible genes (Ballinger and Benzer, (1989) Proc. Natl. Acad. Sci USA 86:9402; Koes et al., (1995) Proc. Natl. Acad. Sci USA 92:8149; Bensen et al., (1995) Plant Cell 7:75). The latter approach may be accomplished in two ways. First, short segments of the instant nucleic acid fragments may be used in polymerase chain reaction protocols in conjunction with a mutation tag sequence primer on DNAs prepared from a population of plants in which Mutator transposons or some other mutation-causing DNA element has been introduced (see Bensen, supra). The amplification of a specific DNA fragment with these primers indicates the insertion of the mutation tag element in or near the maize, rice, or wheat histone acetyltransferase protein gene. Alternatively, the histone acetyltransferase gene may be used as a hybridization probe against PCR amplification products generated from the mutation population using the mutation tag sequence primer in conjunction with an arbitrary genomic site primer, such as that for a restriction enzyme site-anchored synthetic adaptor. With either method, a plant containing a mutation in the endogenous histone acetyltransferase gene can be identified and obtained. This mutant plant can then be used to determine or confirm the natural functon of the maize, rice, or wheat histone acetyltransferase protein gene product.

### EXAMPLES

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### Example 1

### Composition of cDNA Libraries; Isolation and Sequencing of cDNA Clones

cDNA libraries representing mRNAs from various corn, rice and wheat tissues were prepared. The characteristics of the libraries are described in Table 1.

TABLE 1
cDNA Libraries from Corn, Rice and Wheat

| Library | Tissue | Clone |
|---|---|---|
| cep7 | Corn, 7 day old epicotyl, grown in light | cep7.pk0001.a11 |
| wlln | Wheat leaf from 7 day old seedling grown in light* | w11n.pk0003.c1 |
| rlr6 | Rice leaf 15 days after germination and 6 hrs after infection | r1r6.pk0084.c5 |
| | of strain Magaporthe grisa 4360-R-62 (AVR2-YAMO) | |
| wr1 | Wheat root 7 day old seedlings | wr1.pk0045.f4 |

*These libraries were normalized essentially as described in U.S. Pat. No. 5,482,845

cDNA libraries representing the maize, rice and wheat cDNAs were prepared in Uni-ZAP™ XR vectors according to the manufacturer's protocol (Stratagene Cloning Systems, La Jolla, Calif.). Conversion of the Uni-ZAP™ XR libraries into plasmid libraries was accomplished according to the protocol provided by Stratagene. Upon conversion, cDNA inserts were contained in the plasmid vector pBluescript. cDNA inserts from randomly picked bacterial colonies containing recombinant pBluescript plasmids were amplified via polymerase chain reaction using primers specific for vector sequences flanking the inserted cDNA sequences. Amplified insert DNAs were sequenced in dye-primer sequencing reactions to generate partial cDNA sequences (expressed sequence tags or “ESTs”; see Adams, M. D. et al., (1991) Science 252:1651). The resulting ESTs were analyzed using a Perkin Elmer Model 377 fluorescent sequencer.

### Example 2

### Identification and Characterization of cDNA Clones

ESTs encoding maize, rice and wheat histone acetyltransferase protein homologs were identified by conducting BLAST (Basic Local Alignment Search Tool; Altschul, S. F., et al., (1993) J. Mol. Biol. 215:403-410; see also www.ncbi.nlm.nih.gov/BLAST/) searches for similarity to sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The cDNA sequences obtained in Example 1 were analyzed for similarity to all publicly available DNA sequences contained in the “nr” database using the BLASTN algorithm provided by the National Center for Biotechnology Information (NCBI). The DNA sequences were translated in all reading frames and compared for similarity to all publicly available protein sequences contained in the “nr” database using the BLASTX algorithm (Gish, W. and States, D. J. (1993) Nature Genetics 3:266-272) provided by the NCBI. For convenience, the P-value (probability) of observing a match of a cDNA sequence to a sequence contained in the searched databases merely by chance as calculated by BLAST are reported herein as “pLog” values, which represent the negative of the logarithm of the reported P-value. Accordingly, the greater the pLog value, the greater the likelihood that the cDNA sequence and the BLAST “hit” represent homologous proteins.

The BLASTX search using the nucleotide sequence from clone cep7.pk0001.a11 revealed similarity of the protein encoded by the cDNA to Saccharomyces cerevisiae transcription factor GCN5 (EMBL Accession No. X68628; pLog=42.59) and human histone acetyltransferase (GenBank Accession No. U57316; pLog=34.62). SEQ ID NO:1 shows the nucleotide sequence of the entire maize cDNA insert; the deduced amino acid sequence is shown in SEQ ID NO:2. The entire cDNA insert in clone cep7.pk0001.a11 was reevaluated by BLAST, yielding even higher pLog values vs. the Saccharomyces (EMBL Accession No. X68628; pLog=94.06) and human (GenBank Accession No. U57316; pLog=80.92) sequences and an Arabidopsis thaliana histone acetyltransferase (GenBank Accession No. AF037442; pLog=131.00). Sequence alignments and BLAST scores and probabilities indicate that the instant nucleic acid fragment encodes a portion of a maize homolog of a yeast GCN5 transcriptional coactivator protein with histone acetyltransferase activity.

This cDNA clone represents the first EST identified for a maize histone acetyltransferase protein with homology to yeast and human histone acetyltransferase proteins.

### Example 3

### Determination of Further 5′ Sequence of the Maize Homolog of the Yeast GCN5

A Rapid Amplification of cDNA Ends (“5′ RACE”) strategy was used to isolate a 5′ end fragment of the maize homolog of the yeast GCN5. The 5′ RACE was performed with a commercially available 5′ RACE system (BRL) according to manufacturer's protocol. Total RNA was isolated from maize epicotyl obtained from 7 day old seedlings. Based on the nucleotide sequence of cDNA clone cep7.pk0001.a11, three gene-specific primers were designed:

(SEQ ID NO:7)

| | |
|---|---|
| GSP1: | 5′-CTTTGTGAAACCCTGCTTTACAA-3′ |

(SEQ ID NO:8)

| | |
|---|---|
| GSP2: | 5′-CGGAATTCGTTAAGAAATGTGTGAGCCCATCA-3′ |

(SEQ ID NO:9)

| | |
|---|---|
| GSP3: | 5′-CGGAATTCCCCGTGCATGTTGTTTCAAATGATT-3′ |

Two anchor primers as specified in the 5′ RACE protocol provided by the manufacturer were also used:

(SEQ ID NO:10)

5′ RACE Abridged Anchor Primer:
5′-CTTTGTGAAACCCTGCTTTACAA-3′

(SEQ ID NO:11)

Abridged Universal Amplification Primer:
5′-CGGAATTCGTTAAGAAATGTGTGAGCCCATCA-3′

GSP1 was used to prime the first strand cDNA synthesis, thus providing cDNA copies of specific mRNAs corresponding the maize homolog of the yeast GCN5. Following cDNA synthesis, the first strand product was purified from unincorporated dNTPs and residual GSP1 primer using the GlassMaX™ DNA Isolation Spin Cartridge Procedure (BRL). Terminal deoxynucleotidyl transferase was then used to add homopolymeric deoxycytidine tails (oligo-dC tails) to the 3′ end of the cDNA. Tailed cDNA was then amplified by PCR using a nested, gene-specific primer (GSP2) which annealed 3′ to the GSP1 site, and the 5′ RACE Abridged Anchor Primer as specified in the manufacturer's protocol. A portion of the resulting PCR product was used as a template to re-amplify the specific product using another nested, gene-specific primer (GSP3) which annealed 3′ to GSP2 site, and the Abridged Universal Amplification primer. An EcoRI site is encoded within the GSP2 and GSP3 primers; the anchor primers include an SpeI restriction site. The resulting PCR product was therefore digested with EcoRI and SpeI enzymes, subcloned into pBluescript, and sequenced as described in Example 1. One of the 5′ RACE products yielded 271 bp of additional sequence beyond the original 5′-end of the cDNA insert in cDNA clone cep7.pk0001.a11. A contiguous sequence of the maize homolog of the yeast GCN5 consisting of the original cDNA sequence from clone cep7.pk0001.a11 and the additional 5′ RACE sequence is provided in SEQ ID NO:12. A deduced amino acid sequence encoding a portion of the maize homolog of the yeast GCN5 derived from the nucleotide sequence of SEQ ID NO:12 is provided in SEQ ID NO:13. The entire 5′-end sequence of the maize homolog of the yeast GCN5 can be isolated by following a similar strategy.

### Example 4

### Characterization of cDNA Clones Encoding Rice and Wheat Homologs of Arabidopsis thaliana Histone Acetyltransferase

The BLASTX search using the EST sequences from clones r1r6.pk0084.c5 and wr1.pk0045.f4 revealed similarity of the proteins encoded by the cDNAs to a histone acetyltransferase peptide from Arabidopsis thaliana (GenBank Accession No. AF037442). The BLAST results for each of these ESTs are shown in Table 2:

TABLE 2
BLAST Results for Clones Encoding Polypeptides Homologous
to Arabidopsis thaliana Histone Acetyltransferase Proteins

| | BLAST pLog Score |
|---|---|
| Clone | AF037442 |
| rlr6.pk0084.c5 | 35.70 |
| wr1.pk0045.f4 | 13.40 |
| wl1n.pk0003.c2 | 34.52 |

The sequence of a portion of the cDNA insert from clone r1r6.0084.c5 is shown in SEQ ID NO:14; the deduced amino acid sequence of this cDNA is shown in SEQ ID NO:15. The sequence of a portion of the cDNA insert from clone wr1.pk0045.f4 is shown in SEQ ID NO:16; the deduced amino acid sequence of this cDNA is shown in SEQ ID NO:17. The sequence of a portion of the cDNA insert from clone w11n.pk0003.c2 is shown in SEQ ID NO:3; the deduced amino acid sequence of this cDNA is shown in SEQ ID NO:4. BLAST scores and probabilities indicate that the instant nucleic acid fragments encode portions of a histone acetyltransferase protein. These sequences represent the first rice and wheat sequences encoding homologs to an Arabidopsis thaliana histone acetlytransferase protein.

### Example 5

### Expression of Chimeric Genes in Monocot Cells

A chimeric gene comprising a cDNA encoding a maize, rice or wheat histone acetyltransferase protein in sense orientation with respect to the maize 27 kD zein promoter that is located 5′ to the cDNA fragment, and the 10 kD zein 3′ end that is located 3′ to the cDNA fragment, can be constructed. The cDNA fragment of this gene may be generated by polymerase chain reaction (PCR) of the cDNA clone using appropriate oligonucleotide primers. Cloning sites (NcoI or SmaI) can be incorporated into the oligonucleotides to provide proper orientation of the DNA fragment when inserted into the digested vector pML103 as described below. Amplification is then performed in a 100 uL volume in a standard PCR mix consisting of 0.4 mM of each oligonucleotide and 0.3 pM of target DNA in 10 mM Tris-HCl, pH 8.3, 50 mM KCl, 1.5 mM MgCl₂, 0.001% w/v gelatin, 200 mM dGTP, 200 mM dATP, 200 mM dTTP, 200 mM dCTP and 0.025 unit Amplitaq™ DNA polymerase. Reactions are carried out in a Perkin-Elmer Cetus Thermocycler™ for 30 cycles comprising 1 minute at 95° C., 2 minutes at 55° C. and 3 minutes at 72° C., with a final 7 minute extension at 72° C. after the last cycle. The amplified DNA is then digested with restriction enzymes NcoI and SmaI and fractionated on a 0.7% low melting point agarose gel in 40 mM Tris-acetate, pH 8.5, 1 mM EDTA. The appropriate band can be excised from the gel, melted at 68° C. and combined with a 4.9 kb NcoI-SmaI fragment of the plasmid pML103. Plasmid pML103 has been deposited under the terms of the Budapest Treaty at ATCC (American Type Culture Collection, 10801 University Boulevard, Manassas, Va. 20110-2209), and bears accession number ATCC 97366. The DNA segment from pML103 contains a 1.05 kb SalI-NcoI promoter fragment of the maize 27 kD zein gene and a 0.96 kb SmaI-SalI fragment from the 3′ end of the maize 10 kD zein gene in the vector pGem9Zf(+) (Promega). Vector and insert DNA can be ligated at 15° C. overnight, essentially as described (Maniatis). The ligated DNA may then be used to transform E. coli XL1-Blue (Epicurian Coli XL-1 Blue™; Stratagene). Bacterial transformants can be screened by restriction enzyme digestion of plasmid DNA and limited nucleotide sequence analysis using the dideoxy chain termination method (Sequenase™ DNA Sequencing Kit; U.S. Biochemical). The resulting plasmid construct would comprise a chimeric gene encoding, in the 5′ to 3′ direction, the maize 27 kD zein promoter, a cDNA fragment encoding a maize, rice, or wheat histone acetyltransferase protein, and the 10 kD zein 3′ region.

The chimeric gene described above can then be introduced into corn cells by the following procedure. Immature corn embryos can be dissected from developing caryopses derived from crosses of the inbred corn lines H99 and LH132. The embryos are isolated 10 to 11 days after pollination when they are 1.0 to 1.5 mm long. The embryos are then placed with the axis-side facing down and in contact with agarose-solidified N6 medium (Chu et al., (1975) Sci. Sin. Peking 18:659-668). The embryos are kept in the dark at 27° C. Friable embryogenic callus consisting of undifferentiated masses of cells with somatic proembryoids and embryoids borne on suspensor structures proliferates from the scutellum of these immature embryos. The embryogenic callus isolated from the primary explant can be cultured on N6 medium and sub-cultured on this medium every 2 to 3 weeks.

The plasmid, p35S/Ac (obtained from Dr. Peter Eckes, Hoechst Ag, Frankfurt, Germany) may be used in transformation experiments in order to provide for a selectable marker. This plasmid contains the Pat gene (see European Patent Publication 0 242 236) which encodes phosphinothricin acetyl transferase (PAT). The enzyme PAT confers resistance to herbicidal glutamine synthetase inhibitors such as phosphinothricin. The pat gene in p35S/Ac is under the control of the 35S promoter from Cauliflower Mosaic Virus (Odell et al. (1985) Nature 313:810-812) and the 3′ region of the nopaline synthase gene from the T-DNA of the Ti plasmid of Agrobacterium tumefaciens.

The particle bombardment method (Klein et al., (1987) Nature 327:70-73) may be used to transfer genes to the callus culture cells. According to this method, gold particles (1 μm in diameter) are coated with DNA using the following technique. Ten μg of plasmid DNAs are added to 50 μL of a suspension of gold particles (60 mg per mL). Calcium chloride (50 μL of a 2.5 M solution) and spermidine free base (20 μL of a 1.0 M solution) are added to the particles. The suspension is vortexed during the addition of these solutions. After 10 minutes, the tubes are briefly centrifuged (5 sec at 15,000 rpm) and the supernatant removed. The particles are resuspended in 200 μL of absolute ethanol, centrifuged again and the supernatant removed. The ethanol rinse is performed again and the particles resuspended in a final volume of 30 μL of ethanol. An aliquot (5 μL) of the DNA-coated gold particles can be placed in the center of a Kapton™ flying disc (Bio-Rad Labs). The particles are then accelerated into the corn tissue with a Biolistic™ PDS-1000/He (Bio-Rad Instruments, Hercules Calif.), using a helium pressure of 1000 psi, a gap distance of 0.5 cm and a flying distance of 1.0 cm.

For bombardment, the embryogenic tissue is placed on filter paper over agarose-solidified N6 medium. The tissue is arranged as a thin lawn and covered a circular area of about 5 cm in diameter. The petri dish containing the tissue can be placed in the chamber of the PDS-1000/He approximately 8 cm from the stopping screen. The air in the chamber is then evacuated to a vacuum of 28 inches of Hg. The macrocarrier is accelerated with a helium shock wave using a rupture membrane that bursts when the He pressure in the shock tube reaches 1000 psi.

Seven days after bombardment the tissue can be transferred to N6 medium that contains gluphosinate (2 mg per liter) and lacks casein or proline. The tissue continues to grow slowly on this medium. After an additional 2 weeks the tissue can be transferred to fresh N6 medium containing gluphosinate. After 6 weeks, areas of about 1 cm in diameter of actively growing callus can be identified on some of the plates containing the glufosinate-supplemented medium. These calli may continue to grow when sub-cultured on the selective medium.

Plants can be regenerated from the transgenic callus by first transferring clusters of tissue to N6 medium supplemented with 0.2 mg per liter of 2,4-D. After two weeks the tissue can be transferred to regeneration medium (Fromm et al., (1990) Bio/Technology 8:833-839).

### Example 6

### Expression of Chimeric Genes in Dicot Cells

A seed-specific expression cassette composed of the promoter and transcription terminator from the gene encoding the β subunit of the seed storage protein phaseolin from the bean Phaseolus vulgaris (Doyle et al. (1986) J. Biol. Chem. 261:9228-9238) can be used for expression of the instant maize, rice and wheat histone acetyltransferase proteins in transformed soybean. The phaseolin cassette includes about 500 nucleotides upstream (5′) from the translation initiation codon and about 1650 nucleotides downstream (3′) from the translation stop codon of phaseolin. Between the 5′ and 3′ regions are the unique restriction endonuclease sites Nco I (which includes the ATG translation initiation codon), Sma I, Kpn I and Xba I. The entire cassette is flanked by Hind III sites.

A nucleic acid fragment encoding a maize, rice or wheat acetyltransferase proteins may be generated by polymerase chain reaction (PCR) of an appropriate cDNA clone using appropriate oligonucleotide primers. Cloning sites can be incorporated into the oligonucleotides to provide proper orientation of the DNA fragment when inserted into the expression vector. Amplification is then performed, and the isolated fragment is inserted into a pUC18 vector carrying the seed expression cassette.

Soybean embroys may then be transformed with the expression vector comprising sequences encoding a maize, rice, or wheat histone acetyltransferase protein. To induce somatic embryos, cotyledons, 3-5 mm in length dissected from surface sterilized, immature seeds of the soybean cultivar A2872, can be cultured in the light or dark at 26° C. on an appropriate agar medium for 6-10 weeks. Somatic embryos which produce secondary embryos are then excised and placed into a suitable liquid medium. After repeated selection for clusters of somatic embryos which multiplied as early, globular staged embryos, the suspensions are maintained as described below.

Soybean embryogenic suspension cultures can be maintained in 35 mL liquid media on a rotary shaker, 150 rpm, at 26° C. with florescent lights on a 16:8 hour day/night schedule. Cultures are subcultured every two weeks by inoculating approximately 35 mg of tissue into 35 mL of liquid medium.

Soybean embryogenic suspension cultures may then be transformed by the method of particle gun bombardment (Kline et al. (1987) Nature (London) 327:70, U.S. Pat. No. 4,945,050). A Du Pont Biolistic™ PDS 1000/HE instrument (helium retrofit) can be used for these transformations.

A selectable marker gene which can be used to facilitate soybean transformation is a chimeric gene composed of the 35S promoter from Cauliflower Mosaic Virus (Odell et al.(1985) Nature 313:810-812), the hygromycin phosphotransferase gene from plasmid pJR225 (from E. coli; Gritz et al. (1983) Gene 25:179-188) and the 3′ region of the nopaline synthase gene from the T-DNA of the Ti plasmid of Agrobacterium tumefaciens. The seed expression cassette comprising the phaseolin 5′ region, the fragment encoding the maize, rice, or wheat histone acetyltransferase protein homolog and the phaseolin 3′ region can be isolated as a restriction fragment. This fragment can then be inserted into a unique restriction site of the vector carrying the marker gene.

To 50 μL of a 60 mg/mL 1 μm gold particle suspension is added (in order): 5 μL DNA (1 μg/μL), 20 μl spermidine (0.1 M), and 50 μL CaCl₂ (2.5 M). The particle preparation is then agitated for three minutes, spun in a microfuge for 10 seconds and the supernatant removed. The DNA-coated particles are then washed once in 400 μL 70% ethanol and resuspended in 40 μL of anhydrous ethanol. The DNA/particle suspension can be sonicated three times for one second each. Five μL of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post bombardment with fresh media containing 50 mg/mL hygromycin. This selective media can be refreshed weekly. Seven to eight weeks post bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 7

### Expression of Chimeric Genes in Microbial Cells

The cDNAs encoding the instant maize, rice, or wheat histone acetyltransferase protein proteins can be inserted into the T7 E. coli expression vector pET24d (Novagen). Plasmid DNA containing a cDNA may be appropriately digested to release a nucleic acid fragment encoding the histone acetyltransferase protein. This fragment may then be purified on a 1% NuSieve GTG™ low melting agarose gel (FMC). Buffer and agarose contain 10 μg/ml ethidium bromide for visualization of the DNA fragment. The fragment can then be purified from the agarose gel by digestion with GELase™ (Epicentre Technologies) according to the manufacturer's instructions, ethanol precipitated, dried and resuspended in 20 μL of water. Appropriate oligonucleotide adapters may be ligated to the fragment using T4 DNA ligase (New England Biolabs, Beverly, Mass.). The fragment containing the ligated adapters can be purified from the excess adapters using low melting agarose as described above. The vector pET24d is digested, dephosphorylated with alkaline phosphatase (NEB) and deproteinized with phenol/chloroform as decribed above. The prepared vector pET24d and fragment can then be ligated at 16° C. for 15 hours followed by transformation into DH5 electrocompetent cells (GIBCO BRL). Transformants can be selected on agar plates containing 2xYT media and 50 μg/mL kanamycin. Transformants containing the gene are then screened for the correct orientation with respect to pET24d T7 promoter by restriction enzyme analysis.

Clones in the correct orientation with respect to the T7 promoter can be transformed into BL21(DE3) competent cells (Novagen) and selected on 2xYT agar plates containing 50 μg/ml kanamycin. A colony arising from this transformation construct can be grown overnight at 30° C. in 2xYT media with 50 μg/mL kanamycin. The culture is then diluted two fold with fresh media, allowed to re-grow for 1 h, and induced by adding isopropyl-thiogalactopyranoside to 1 mM final concentration. Cells are then harvested by centrifugation after 3 h and re-suspended in 50 μL of 50 mM Tris-HCl at pH 8.0 containing 0.1 mM DTT and 0.2 mM phenyl methylsulfonyl fluoride. A small amount of 1 mm glass beads can be added and the mixture sonicated 3 times for about 5 seconds each time with a microprobe sonicator. The mixture is centrifuged and the protein concentration of the supernatant determined. One μg of protein from the soluble fraction of the culture can be separated by SDS-polyacrylamide gel electrophoresis. Gels can be observed for protein bands migrating at the expected molecular weight.

### Example 8

### Evaluating Compounds for Their Ability to Inhibit the Activity of Maize, Rice and Wheat Histone Acetyltransferase Proteins

The maize, rice and wheat acetyltransferase proteins described herein may be produced using any number of methods known to those skilled in the art. Such methods include, but are not limited to, expression in bacteria as described in Example 6, or expression in eukaryotic cell culture, in planta, and using viral expression systems in suitably infected organisms or cell lines. The instant polypeptides may be expressed either as mature forms of the proteins as observed in vivo or as fusion proteins by covalent attachment to a variety of enzymes, proteins or affinity tags. Common fusion protein partners include glutathione S-transferase (“GST”), thioredoxin (“Trx”), maltose binding protein, and C- and/or N-terminal hexahistidine polypeptide (“(His)₆”). The fusion proteins may be engineered with a protease recognition site at the fusion point so that fusion partners can be separated by protease digestion to yield intact mature polypeptides. Examples of such proteases include thrombin, enterokinase and factor Xa. However, any protease can be used which specifically cleaves the peptide connecting the fusion protein and the maize, rice, or wheat histone acetyltransferase polypeptide.

Purification of the instant polypeptides, if desired, may utilize any number of separation technologies familiar to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography and affinity chromatography, wherein the affinity ligand represents a substrate, substrate analog or inhibitor. When the polypeptides are expressed as fusion proteins, the purification protocol may include the use of an affinity resin which is specific for the fusion protein tag attached to the expressed polypeptide or an affinity resin containing ligands which are specific for the polypeptide. For example, polypeptide may be expressed as a fusion protein coupled to the C-terminus of thioredoxin. In addition, a (His)₆ peptide may be engineered into the N-terminus of the fused thioredoxin moiety to afford additional opportunities for affinity purification. Other suitable affinity resins could be synthesized by linking the appropriate ligands to any suitable resin such as Sepharose-4B. In an alternate embodiment, a thioredoxin fusion protein may be eluted using dithiothreitol; however, elution may be accomplished using other reagents which interact to displace the thioredoxin from the resin. These reagents include P-mercaptoethanol or other reduced thiol. The eluted fusion protein may be subjected to further purification by traditional means as stated above, if desired. Proteolytic cleavage of the thioredoxin fusion protein and the maize, rice and wheat histone acetyltransferase proteins may be accomplished after the fusion protein is purified or while the protein is still bound to the ThioBond™ affinity resin or other resin.

Crude, partially purified or purified polypeptide, either alone or as a fusion protein, may be utilized in assays for the evaluation of compounds for their ability to inhibit the activity of the maize, rice and wheat histone acetyltransferase proteins disclosed herein. Assays may be conducted under well known experimental conditions which permit optimal activity. An example of an in vitro assay for histone acteyltransferase activity may be found in Brownell, J. E. and Allis, C. D. (1995) Proc. Natl. Acad. Sci. USA 92:6364-6368. The skilled artisan is well aware of simple modifications that could be made to the published protocol that would afford detection of inhibitors of histone acteyltransferase activity.

What is claimed is:

1. An isolated polynucleotide comprising:
(a) a first nucleotide sequence encoding a polypeptide having the activity of histone acetyltransferase, wherein the first nucleotide sequence and the nucleotide sequence of SEQ ID NO:12 have at least 80% sequence identity, or
(b) a second nucleotide sequence comprising a complement of the first nucleotide sequence, wherein the first and second nucleotide sequences contain the same number of nucleotides and are 100% complementary in a pairwise alignment.

2. The polynucleotide of claim 1, wherein the first nucleotide sequence and the nucleotide sequence of SEQ ID NO:12 have at least 85% sequence identity.

3. The polynucleotide of claim 1, wherein the first nucleotide sequence and the nucleotide sequence of SEQ ID NO:12 have at least 90% sequence identity.

4. The polynucleotide of claim 1, wherein the first nucleotide sequence and the nucleotide sequence of SEQ ID NO:12 have at least 95% sequence identity.

5. The polynucleotide of claim 1, wherein the first nucleotide sequence comprises the nucleotide sequence of SEQ ID NO:12.

6. A vector comprising the polynucleotide of claim 1.

7. A recombinant DNA construct comprising the polynucleotide of claim 1 operably linked to a regulatory sequence.

8. A method for transforming a cell comprising transforming a cell with the polynucleotide of claim 1.

9. A cell comprising the recombinant DNA construct of claim 7.

10. A method for producing a plant comprising transforming a plant cell with the polynucleotide of claim 1 and regenerating a plant from the transformed plant cell.

11. A plant comprising the recombinant DNA construct of claim 7.

12. A seed comprising the recombinant DNA construct of claim 7.
